(19) United States
(12) Patent Application Publication
(10) Pub. No.: US 2025/0288809 A1
(43) Pub. Date: Sep. 18, 2025

(54) AUTOMATED DETECTION OF LEAD CONFIGURATIONS FOR IMPLANTABLE MEDICAL DEVICES

(71) Applicant: THE ALFRED E. MANN FOUNDATION FOR SCIENTIFIC RESEARCH, VALENCIA, CA (US)

(72) Inventor: Anthony Ng, Santa Clarita, CA (US)

(21) Appl. No.: 19/079,170

(22) Filed: Mar. 13, 2025

Related U.S. Application Data
(60) Provisional application No. 63/565,419, filed on Mar. 14, 2024.

(51) Int. Cl. A61N 1/36; A61N 1/372

(52) U.S. Cl. CPC A61N 1/36132; A61N 1/36125; A61N 1/36142; A61N 1/37258

(57) ABSTRACT

The present disclosure generally pertains to implantable stimulation systems. More specifically, the present disclosure provides systems and methods for automatically detecting the configuration of stimulation leads and optionally determining if any such leads are electrically defective. The method includes iteratively applying stimulation through anode-cathode pairs of electrode contacts disposed on one or more leads, measuring the impedance values between each respective pair, and comparing the impedance values to one or more threshold values to determine if the leads are unilateral or bilateral.

### CROSS-REFERENCE TO RELATED APPLICATION

This application claims priority to, and the benefit of, U.S. Provisional Application No. 63/565,419, filed Mar. 14, 2024, the entire content of which is hereby incorporated by reference.

### FIELD OF THE DISCLOSURE

The present disclosure generally pertains to implantable stimulation systems. More specifically, the present disclosure provides systems and methods for automatically detecting the configuration of stimulation leads connected to an implantable medical device, optionally determining if any such stimulation leads are electrically defective, and displaying the detected configuration on a graphical user interface.

### BACKGROUND

Various types of devices have been developed for implantation into the human body to provide various types of health-related therapies and/or monitoring. Examples of such devices, generally known as implantable medical devices (“IMDs”), include cardiac pacemakers, cardioverter/defibrillators, cardiomyostimulators, various physiological stimulators including nerve, muscle, and deep brain stimulators, various types of physiological monitors, and drug delivery systems, just to name a few. For the purposes of this application, reference will be made to implantable neurostimulators, such as implantable pulse generators (“IPGs”), it being understood that the principles described herein may have applicability to other implantable medical devices as well.

IPGs are often used in the context of neuromodulation therapy, and, in general, comprise a hermetically sealed housing, typically made of a biocompatible metal such as a titanium alloy, that houses stimulation circuitry, and a header, typically made of an epoxy housing, which is mounted on the IPG housing. The header includes at least one receptacle adapted to receive a connector associated with the proximal end of a stimulation lead, and feedthrough pins extending from the header into the IPG housing to electrically couple the stimulation lead with the IPG stimulation circuitry.

Stimulation leads associated with such IPGs typically include a lead body extending between the proximal lead end and a distal lead end and incorporate one or more exposed electrode contacts located at or near the distal lead end. One or more electrical conductors extend through the lead body from the connector at the proximal end and couple with a respective electrode contact located at the distal lead end or along a section of the lead body. Each electrode contact is typically electrically isolated from any other electrical conductors and may be carried by the electrically insulative body of a neural interface (e.g., stimulation electrode) that is configured to interface with an anatomical target, such as a peripheral nerve. In various applications, the electrode contacts may be used for transmitting electrical stimulation from the IPG to the anatomical target, or for sensing electrical activity in the vicinity of an anatomical target.

Implantable stimulation leads and stimulation electrodes come in a variety of forms to accommodate different applications. For example, a non-exhaustive list of neural interfaces known in the prior art includes: nerve cuff electrodes (“nerve cuff”) as disclosed in U.S. Pat. No. 9,283,394; helical electrodes as disclosed in U.S. Pat. No. 4,573,481; paddle electrodes (or “electrode arrays”) as disclosed in U.S. Pat. Pub. 2012/0209285A1; and linear electrodes (or “percutaneous electrodes”) as disclosed in U.S. Pat. No. 8,650,747. For purposes of this disclosure, a “neural interface” or “stimulation electrode” may refer to a part (e.g., the distal part) of a stimulation lead that has one or more electrode contacts. Although the present disclosure primarily refers to nerve cuffs, it will be understood that other types of neural interfaces (e.g., helical electrodes, paddle electrodes, linear electrodes, etc.) can also be used.

Electrical stimulation can be provided to one or more nerves to treat one or more medical conditions, and, in certain applications, it can be beneficial to stimulate multiple nerves to effectively treat a given condition. Stimulation of the hypoglossal or vagus nerves, for example, could be applied bilaterally, e.g. stimulating simultaneously both the right and left hypoglossal nerves, or unilaterally, e.g., stimulating only one of the right or left hypoglossal nerves.

Damaged or malfunctioning stimulation leads are a concern for patients and physicians alike. If a damaged or malfunctioning lead is not identified, the newly-implanted system could, at best, fail to deliver the desired treatment, cause undue pain or discomfort to the patient, and may require an additional surgery to explant and replace. In many cases, it can be difficult for a physician to accurately identify a malfunctioning lead, and this is especially true when a stimulation system includes multiple leads, or when a malfunctioning lead still retains some, but not all, of its ability to transmit electrical current.

Stimulation lead configurations can vary with respect to the type, quantity, and/or distribution of attached leads, stimulation electrodes, and electrode contacts. For example, a simple lead configuration for providing unilateral nerve stimulation might include only one stimulation lead proximally connected to an IPG and distally connected to a neural interface, such as, e.g., a nerve cuff. However, a more complex lead configuration (such as, e.g., that which is disclosed in U.S. Pat. No. 11,081,222 to Livanova), might include a plurality of leads, or may include a bifurcated lead, for stimulating one or more anatomical targets. In various systems, each lead may be coupled to a respective IPG receptacle, or adapters could be used to couple a plurality of leads to a single IPG receptacle.

Occasionally, an IPG may need to be replaced sometime after the initial implantation procedure due to, e.g., battery depletion, technical malfunction, or otherwise. In such cases, the old IPG is excised and replaced with a new IPG that must be programmatically configured to work synergistically with the formerly implanted leads, and this is typically carried out using a clinician programming device to manually select the appropriate device settings for the new IPG. However, it can be difficult for a surgeon to visually identify the configuration of the formerly implanted stimulation leads from the vantage point of the IPG implantation site, and, because many IPGs are capable of accommodating a variety of lead configurations, the process of manually selecting the proper configuration on the clinician programming device has a high propensity for error.

Processes for manufacturing implantable medical devices (IMDs) continually stand to be improved for the purposes of minimizing product size, lowering production costs, increasing product longevity, decreasing surgical times, and improving product performance, among others. This is also true for IMDs such as neurostimulation systems, which may include implantable pulse generators (IPGs) and stimulation leads.

### SUMMARY

Discussed herein are stimulation systems and associated methods for automatically detecting the configuration of one or more attached stimulation leads to determine, for example, if the one or more attached stimulation leads are configured for unilateral or bilateral stimulation, and such systems may also be capable of automatically detecting if one or more attached leads are bifurcated or coupled via an adapter. Optionally, systems and methods described herein may also be capable of detecting if any of a plurality of electrically conductive elements within the leads are electrically defective, and information pertaining to the detected lead configuration (and/or the lead functionality) may optionally be transmitted to an external controller and displayed on a graphical user interface.

In one aspect, the present disclosure provides a stimulation system, which may include an IPG, that is capable of automatically detecting the configuration of one or more attached stimulation leads. The IPG may include a housing containing stimulation circuitry and a header lead connector that defines a receptacle or receptacles configured to receive the proximal end of one or more stimulation leads. Each of the one or more stimulation leads may include a respective distal end in connection with an electrode, such as, for example, a nerve cuff, a paddle electrode, or linear/percutaneous electrode, and each electrode may include a plurality of electrode contact.

In various embodiments, the electrode contacts may function as either anodes or cathodes for the transmission of electrical current from an IPG. For example, some embodiments may include stimulation leads being configured to provide bipolar stimulation, wherein a pair of electrode contact within the same electrode, e.g. a nerve cuff or a paddle electrode, form an anode-cathode pair, and the IPG may be programmatically configured such that any electrode contact within the electrode can be programmed to function as either a cathode or an anode. Some electrode contacts may be unused and not function as either a cathode or anode. In some embodiments, the housing of the IPG, which is made from electrically conductive material, can function as the anode or “indifferent” contact. This mode of stimulation, where the housing serves as an indifferent electrode, is called unipolar or monopolar stimulation. Although the IPG housing is capable of being selected as an indifferent electrode to stimulate in a unipolar mode, the IPG may also be capable of being programmed to stimulate in a bipolar mode. That is, at least one electrode contact is chosen as a cathode and at least one electrode contact is chosen as an anode contact. In the latter case, each electrode contact in the neural interface, e.g., nerve cuff, paddle electrode or linear/percutaneous electrode, may be selected as a cathode electrode contact. Some electrode contacts within the electrode may be unused and not functioning as either cathode or anode.

In another aspect, the present disclosure provides a method for automatically detecting the configuration of stimulation leads in connection with an IPG.

In some embodiments, the stimulation lead may be bifurcated such that a primary lead body branches into two or more sub-leads, and each sub-lead may be distally connected to respective electrodes each containing a plurality of electrode contacts. In other cases, two or more stimulation leads may be electrically coupled to a single receptacle on an IPG header through the use of one or more adaptors.

Embodiments for stimulation systems in accordance with the present disclosure are not limited to any particular type of stimulation electrode, nor are they limited to any particular configuration of stimulation leads. For example, a stimulation lead in accordance with the present disclosure may include any one or a combination of: nerve cuff, helical electrodes, linear electrodes, paddle electrodes, fine-wire electrodes, electrode arrays, or otherwise. Further, such systems could include one or a plurality of stimulation leads being configured to transmit electrical stimulation unilaterally (i.e. to only one side of the body) or bilaterally (i.e. to two or more sides of the body).

In various embodiments, a method for detecting the configuration of stimulation leads in connection with stimulation systems of the present disclosure involves sequentially measuring the bio-impedance levels between various anode-cathode pairs of electrode contacts, grouping anode-cathode pairs based on the measured impedance, and comparing the measured impedance values to at least one threshold value for the purpose of determining if the connected stimulation leads are configured for unilateral or bilateral stimulation.

In some embodiments, a first threshold impedance value may be, for example, about 4,000 Ohms, wherein when a measured impedance between a first anode and a first cathode is less than 4,000 Ohms, the first anode and the first cathode may be determined to be on the same stimulation lead. Further, when a measured impedance between the first anode and a second cathode is greater than 4,000 Ohms, the first anode and the second cathode may be determined to be on separate stimulation leads, and therefore, the stimulation leads may be determined to be configured for bilateral stimulation, with two lead bodies and two electrodes. However, if the first anode, when paired with any other cathode, does not have a measured impedance that is greater than 4,000 Ohms, the detected configuration of the stimulation leads may be determined to be unilateral with only one lead body and one electrode, e.g., a single nerve cuff.

In other embodiments, a different threshold value may be used. For example, certain embodiments may include one or more threshold values between 3,000 to 6,000 Ohms, between 4,000 to 8,000 Ohms, between 7,000-12,000 Ohms, between 10,000 to 20,000 ohms, and/or greater than 30,000 Ohms.

### BRIEF DESCRIPTION OF THE DRAWINGS

FIG. 1 is a plan view of a stimulation system in accordance with one embodiment of the present disclosure.

FIG. 2A is a perspective view of stimulation lead having six electrode contacts.

FIG. 2B shows a view of the nerve cuff of the stimulation lead of FIG. 2A in an unfurled state showing the six electrode contacts within the inner surface of the nerve cuff.

FIG. 3A is a schematic view of least some stimulation circuitry of an IPG, and of left and right nerve cuff, when two electrode contacts on a same nerve cuff are activated.

FIG. 3B differs from the schematic view of FIG. 3A in that two electrode contacts on different nerve cuff are activated.

FIG. 4 is a view of another embodiment for a stimulation system including two simulation leads being connected to an IPG via a bifurcated lead.

FIG. 5 shows a view of the system showing an IPG, a splitter adaptor, and two stimulation leads having nerve cuffs on each lead.

FIGS. 6A and 6B are flow diagrams providing a method, according to some examples, for automatically detecting the configuration of one or more stimulation leads and for determining if the one or more stimulation leads are unilateral or bilateral.

FIG. 7 is a histogram showing an exemplary distribution of impedance values measured across electrode contact pairs within a dual (or bilateral) lead.

FIG. 8 is a histogram showing another exemplary distribution of impedance values measured across electrode contact pairs within a dual (or bilateral) lead.

FIG. 9 is a histogram showing another exemplary distribution of impedance values measured across electrode contact pairs within a dual (or bilateral) lead that includes electrical defects.

FIG. 10 is a histogram showing a distribution of impedance values measured across electrode contact pairs within a single (or unilateral) lead.

FIG. 11 is histogram showing a distribution of impedance values measured across electrode contact pairs within a single (or unilateral) lead that includes electrical defects.

### DETAILED DESCRIPTION

The detailed description set forth below in connection with the appended drawings is intended as a description of various nonlimiting and non-exhaustive examples and configurations and is not intended to represent the only examples and configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Hereinafter, example embodiments will be described in more detail. The subject matter of the present disclosure, however, may be embodied in various different forms, and should not be construed as being limited to only the illustrated and described embodiments herein. Rather, these embodiments are provided as examples so that this disclosure will be thorough and complete, and will fully convey the aspects and features of the present disclosure to those skilled in the art. Accordingly, processes, elements, and techniques that are not necessary to those having ordinary skill in the art for a complete understanding of the aspects and features of the present disclosure may not be described. Unless otherwise noted, like reference numerals denote like elements throughout the attached drawings and the written description, and thus, descriptions thereof will not be repeated. In the drawings, the relative sizes of elements, layers, and regions may be exaggerated for clarity.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

It will be understood that when an element or layer is referred to as being “on,” “connected to,” or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, acts, operations, elements, and/or components, but do not preclude the presence or addition of one or more other features, integers, acts, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

As used herein, the terms “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.” As used herein, the terms “use,” “using,” and “used” may be considered synonymous with the terms “utilize,” “utilizing,” and “utilized,” respectively.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic or electric devices and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of these devices may be formed on one integrated circuit (IC) chip or on separate IC chips. Further, the various components of these devices may be implemented on a flexible printed circuit film, a tape carrier package (TCP), a printed circuit board (PCB), or formed on one substrate. Further, the various components of these devices may be a process or thread, running on one or more processors, in one or more computing devices, executing computer program instructions and interacting with other system components for performing the various functionalities described herein. The computer program instructions may be stored in a memory which may be implemented in a computing device using a standard memory device, such as, for example, a random-access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the spirit and scope of the example embodiments of the present disclosure.

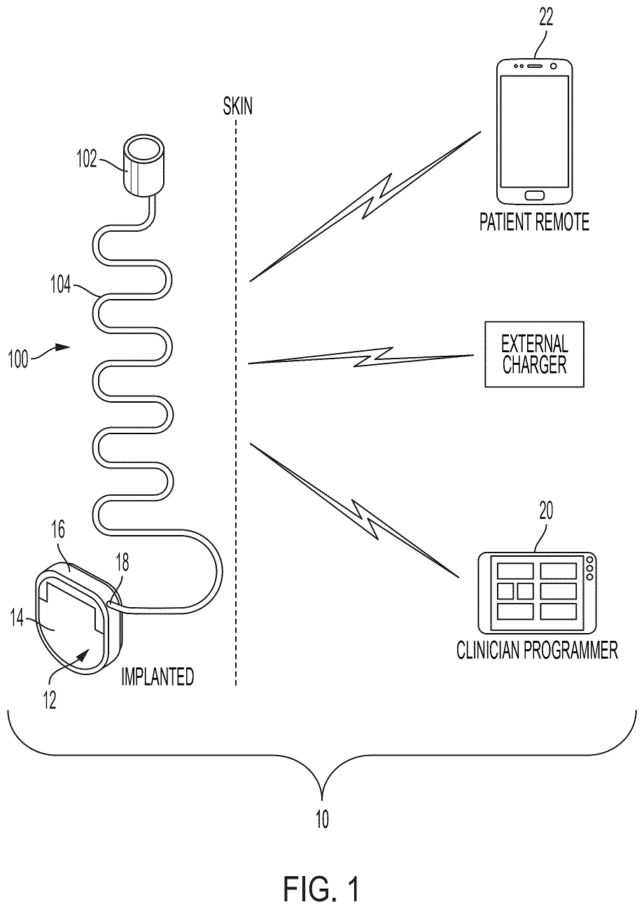

Referring to FIG. 1, a stimulation system 10 in accordance with one embodiment may include an implantable stimulator 12 such as an implantable pulse generator (“IPG”) 12 that includes a housing 14 containing stimulation circuitry. A clinician's programming unit 20, a patient programmer, a patient remote 22, and/or an IPG charger may also be provided in some instances and may be communicatively (e.g., wirelessly communicatively) connectable to the IPG 12, for example, by a Bluetooth or Bluetooth Low Energy (BLE) connection. Suitable IPGs, clinician's programming units and patient remotes are described, for example, in U.S. Pat. Pub. No. 2022/0313987A1.

In some examples, the IPG 12 includes (e.g., within the housing 14) a processor (e.g., at least one processing circuit) and a memory. The processor may be operatively coupled to other components of the IPG, such as the stimulation circuitry and the memory, and be configured to control at least some processes of the IPG 12, including any combination of processes of the IPG 12 disclosed herein. The memory may have stored therein instructions (e.g., computer readable instructions) that, when executed by the processor, cause the processor to perform one or more processes of the IPG 12, including any combination of processes of the IPG 12 disclosed herein. The IPG 12 may include other components, such as a transmitter (e.g. wireless transmitter), a receiver (e.g. wireless receiver), and/or a transceiver (e.g. wireless transceiver) configured to allow the IPG 12 to communicate (e.g. wirelessly communicate) with external devices, such as the clinician's programming unit 20, the patient programmer, the patient remote 22, and/or the IPG charger.

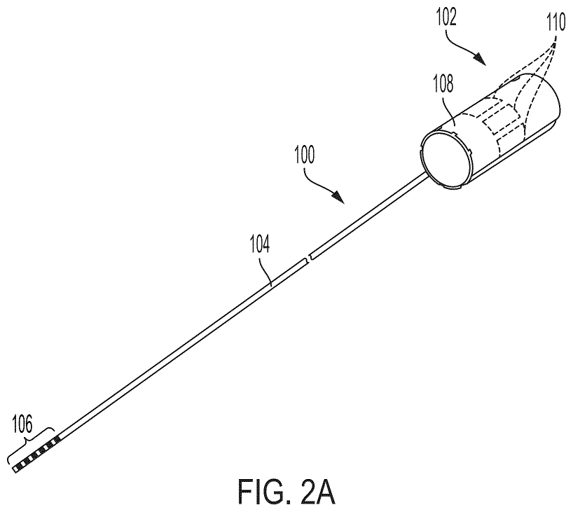

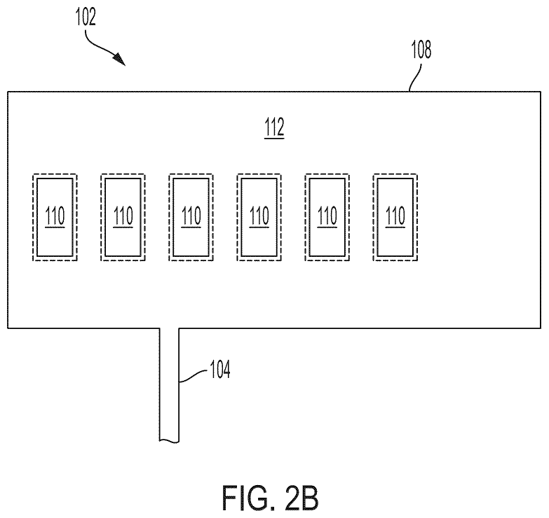

FIG. 2A shows a stimulation lead 100 having a nerve cuff 102, as shown in FIG. 1. A connector 106 is disposed on a proximal end of a lead body 104 of the stimulation lead 100 and is configured to mate with a connector receptacle 18 on a header 16 of the IPG 12 (FIG. 1). The header 16 may be attached to the housing 14 of the IPG 12. The nerve cuff 102 is attached to the distal end of lead body 104. The nerve cuff 102 includes cuff body 108, which comprises a pliable, electrically insulative cuff substrate material, and the substrate material has a naturally coiled shape wherein a front inner surface defines an inner lumen for receiving a nerve. Following surgical implantation, as will be further described, the cuff body 108 is configured to receive a nerve within the inner cuff lumen, and electrical stimulation is passed to the nerve through electrode contacts 110, which are secured to the front inner surface 112 (as shown in FIG. 2B).

Referring to FIG. 2B, an enlarged view of one embodiment for a nerve cuff 102 is shown in an unfurled state. The nerve cuff 102 comprises a biocompatible, pliable and electrically insulative cuff body substrate material 108, such as, e.g., silicone, and a plurality of electrode contacts 110 carried by an inner surface 112 of the cuff body 108. In the illustrated embodiment, six electrode contacts 110 are disposed on the nerve cuff inner surface 112 and are spaced apart linearly along a width direction of the cuff body 108. In other embodiments, nerve cuffs 102 (or another type of electrode) incorporated into stimulation systems of the present disclosure may carry more or fewer electrode contacts 110 than shown, and the electrode contacts 110 may be distributed in a manner that differs from that which is shown in FIG. 2B.

Comparing FIGS. 2A and 2B, the unfurled nerve cuff 102 in FIG. 2B is illustrated to include six electrode contact 110, and each of these electrode contacts 110 corresponds with a respective terminal on the connector 106 on the proximal end of the lead body 104. An electrical conductor (not shown) extends through the lead body 104 from each terminal on the connector 106 to a respective electrode contact 110. In the example shown in FIGS. 2A and 2B, six electrical conductors (e.g., electrical wires) may extend through the lead body 104 respectively from the six terminals on the connector 106 to the six electrode contacts 110 on the nerve cuff 102.

The header 16 may include a plurality of feedthrough pins configured to respectively electrically connect to the plurality of terminals on the connector 106 when the connector 106 is inserted into the header 16. The feedthrough pins may extend into the housing 14 and be electrically coupled to electronics within the housing 14, such as to the stimulation circuitry of the IPG 12. The stimulation circuitry may include components configured to controllably provide stimulation (e.g., an electric current or an electric voltage) to the feedthrough pins. When the stimulation lead 100 is coupled to the IPG 12, the stimulation can thus be provided to the nerve cuff 102 through the terminals of the connection portion 106 and through the electrical conductors in the lead body 104 to the electrode contacts 110.

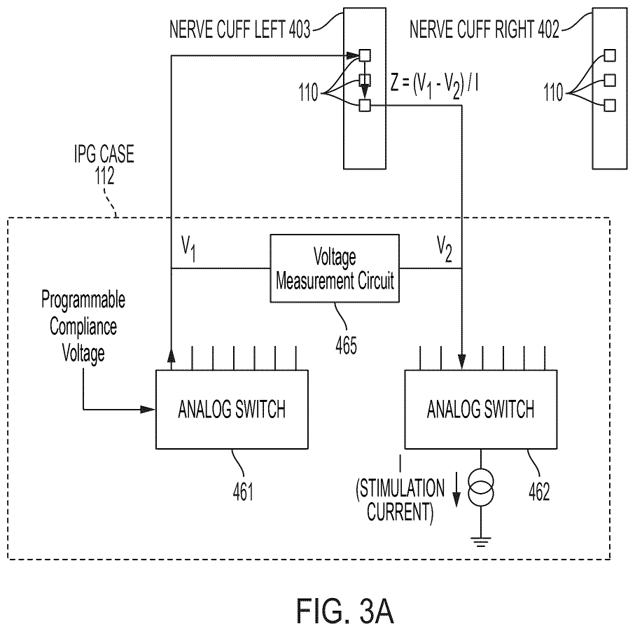

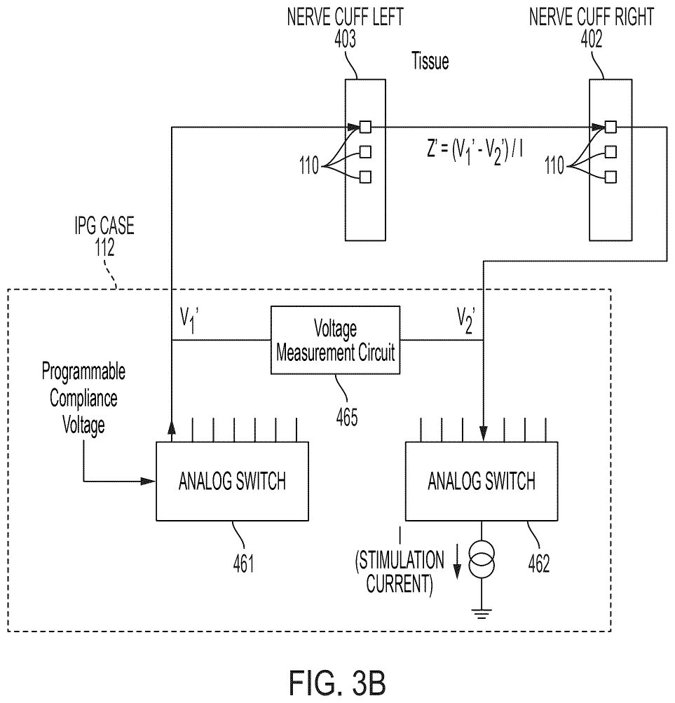

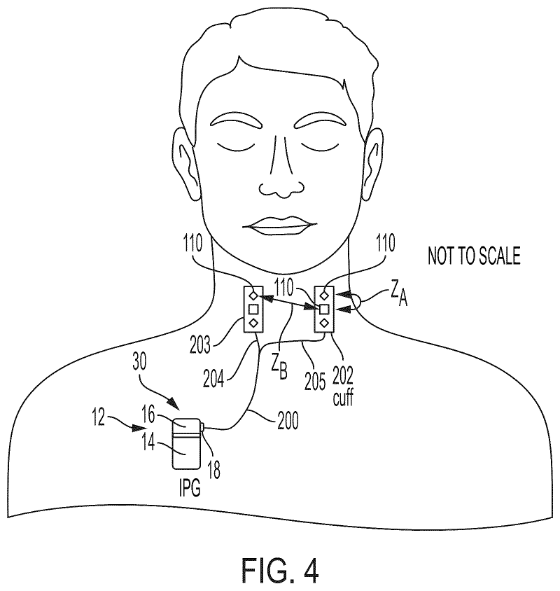

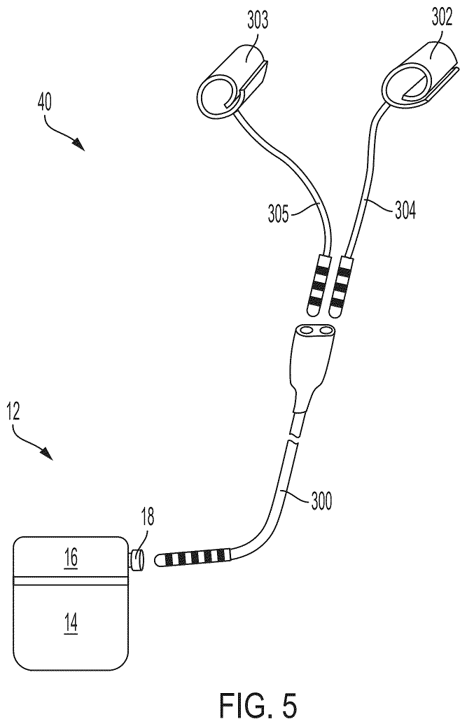

The stimulation circuitry may be configured to select, and electrically connect to, a pair of feedthrough pins (and a corresponding pair of electrode contacts 110), and to generate and provide the stimulation to the pair of feedthrough pins (and the corresponding pair of electrode contacts 110). The IPG 12 (e.g., the stimulation circuitry) may also include an impedance measurement circuitry that is configured to measure the impedance between the pair of electrode contacts 110 and/or between the pair of feedthrough pins. FIGS. 3A and 3B schematically depict some features of the stimulation circuitry and impedance measurement circuitry, according to some examples, when the IPG is coupled to a stimulation lead having two separate nerve cuffs. This stimulation lead may therefore be the one shown in FIG. 4 or 5, as described in more detail below. However, other stimulation leads, such as the one shown in FIGS. 1, 2A, and 2B, may also be used. In FIG. 3A, the stimulation circuitry is electrically coupled, and provides stimulation, to a pair of electrode contacts on a same one (the left) nerve cuff, while in FIG. 3B the stimulation circuitry is electrically coupled, and provides stimulation, to a pair of electrode contacts on separate nerve cuffs.

Referring to FIGS. 3A and 3B, the stimulation circuitry may include a first switch 461 and a second switch 462. The first switch 461 may be coupled to a current source (not shown) and configured to selectively provide a current received from the current source to any one of multiple (e.g., all) electrode contacts 110. For example, the first switch 461 may be configured to selectively couple to (e.g., selectively switch between) any one of the feedthrough pins of the IPG 112. The second switch 462 may be coupled to a current sink (e.g., a ground) and configured to selectively receive a current from any one of multiple (e.g., all) electrode contacts 110 and to provide the received current to the current sink. For example, the second switch 462 may be configured to selectively couple to (e.g., selectively switch between) any one of the feedthrough pins of the IPG 112.

The impedance measurement circuitry may include a voltage measurement circuit 465 that is configured to measure a first voltage or current provided by the current source and a second voltage or current received by the current sink (or to measure a difference between the first voltage or current and the second voltage or current). For example, the voltage measurement circuit 465 may be configured to measure the voltage or current at each of the feedthrough pin that the first switch 461 provides the current to and the feedthrough pin that the second switch 462 receives current from. The impedance measurement circuitry may be configured to determine (e.g., calculate) the measure the impedance between the pair of electrode contacts 110 and/or between the pair of feedthrough pins based on the measurement(s) obtained by the voltage measurement circuit 465.

The processor of the IPG 12 may be operatively coupled to the components of the stimulation circuitry and impedance measurement circuitry (e.g., to the current source, the current sink, the first switch 461, the second switch 462, and the voltage measurement circuit 465), and may be configured to control operations of such components to control and monitor the stimulation provided to the feedthrough pins and electrode contacts 110.

Referring to FIG. 4, an implanted stimulation system 30 configured for providing bilateral stimulation is shown. The bilateral stimulation system 30 includes an IPG 12 in proximal connection with a bifurcated stimulation lead 200, which includes two sub-leads 204 and 205 each respectively being distally connected to a stimulation electrode 202 and 203, such as, e.g., a nerve cuff or a linear electrode, and the stimulation electrodes 202 and 203 are configured to provide stimulation on respective sides of the body, i.e. bilaterally. In the example of FIG. 4, the two sub-leads 204 and 205 are connected to (e.g. branch or split off from) a distal end of a common (or main) lead. The common lead is connected, at a proximal end of the common lead, to the header 16 of the IPG 12 via the header receptacle 18. The stimulation electrodes 202 and 203 may each include a plurality of electrode contacts 110. In the example of FIG. 4, each of the stimulation electrodes 202 and 203 includes 3 electrode contacts 110. In a similar manner as described above for the stimulation lead 100 of FIGS. 2A-2B, the connection portion of the stimulation lead 200 may include a plurality of terminals configured to electrically connect to the feedthrough pins in the header 16 and electrically coupled to the electrode contacts 110 through the lead body.

Referring to FIG. 5, in certain circumstances, two or more stimulation leads 304 and 305 may need to connect to a single IPG connection port and, thus, an adaptor (or “splitter”) 300 may be used to couple two or more leads 304, 305 to the IPG header receptacle 18. The adaptor 300 may include, at a proximal end, a connector having a plurality of terminals configured to electrically connect to the feedthrough pins in the header 16 and, at a distal end, multiple lead receptacles (e.g., two lead receptacles in the depicted example) respectively configured to receive proximal ends of the two or more leads 304, 305. The adaptor 300 may include a plurality of electrical conductors (e.g., electrical wires), each coupled between one of the terminals of the connection portion and one of the lead receptacles. For example, a first set (e.g., one half) of the plurality of electrical conductors may be coupled to one of the multiple lead receptacles, and a second set (e.g., the other half) of the plurality of electrical conductors may be coupled to another one of the multiple lead receptacles. In the example of FIG. 5, the connector includes six terminals, the adaptor 300 includes two lead receptacles, and each of leads 304 and 305 includes three terminals at a proximal end thereof that is configured to couple with three electrical conductors in a respective one of the two lead receptacles.

Similar to the bifurcated lead configuration shown in FIG. 4, the multi-lead 304, 305 and adaptor 300 configuration shown in FIG. 5 may be used to provide stimulation bilaterally (i.e. to both sides of the body, such as to the two sagittal sides of the body). In addition the two leaded systems shown in FIGS. 4 and 5 may be used to target two different nerves, for example one target may be the hypoglossal nerve and the second target may be the cervical vagus nerve in the neck area. It is possible that both nerve cuffs may be positioned on the same side of the body and, therefore, positioned in a “unilateral” manner. However, for purposes of this disclosure, “unilateral stimulation” may refer to examples where there is a single lead body used by the stimulation system. For purposes of this disclosure, “bilateral stimulation” may refer to examples where two lead bodies, each having an electrode at the end of the lead body, with each electrode placed on the same nerve, e.g., both cervical vagus nerves, symmetrically on either side of the patient's body. The system of FIG. 1 is chosen for a pure unilateral stimulation or singular target stimulation.

As addressed above, in some instances, it may be necessary for an IPG to be replaced some time after the initial implantation procedure due to depletion of the IPG battery, IPG technical malfunction, or otherwise. In such cases, a surgeon will disconnect the old IPG from the existing stimulation lead or leads, explant the old IPG, implant a new IPG, and proximally connect the existing stimulation leads to the IPG header. The stimulation lead or leads already implanted are generally not explanted but left in place.

However, with reference to FIGS. 4 and 5, implementations of bifurcated leads 204, 205, 304 and 305, and a lead adaptor or lead splitter 300 can make it difficult for a surgeon to accurately discern how the leads are configured “upstream” from the IPG implantation site, and, for example, a surgeon may not be able to discern if the formerly implanted leads are configured for unilateral or bilateral stimulation. For example, when explanting the IPG, the surgeon may only see the proximal end of the stimulation lead 100, 200, or only the proximal end of the lead adaptor 300, and may be unable to see whether the remainder of the lead remains unitary, bifurcates, or is coupled (e.g., via a lead adaptor or splitter) to multiple other leads. Following replacement with the new IPG 12, the physician would typically be responsible for inputting the present lead configuration into a clinician programming device 20, thereby configuring the new IPG 12 to work synergistically with the formerly implanted leads. However, uncertainty as to the configuration of the formerly implanted leads, and/or user error in inputting proper values into the clinician programmer 20 can cause device malfunction and/or inefficient stimulation. As such, the present disclosure provides a method by which stimulation systems 10, 30, or 40 can automatically detect the configuration of any stimulation leads attached thereto for the purpose of determining if the attached leads are configured for unilateral stimulation (corresponding to a unilateral configuration) or bilateral stimulation (corresponding to a bilateral configuration) and automatically configure the IPG 12 for proper workflow.

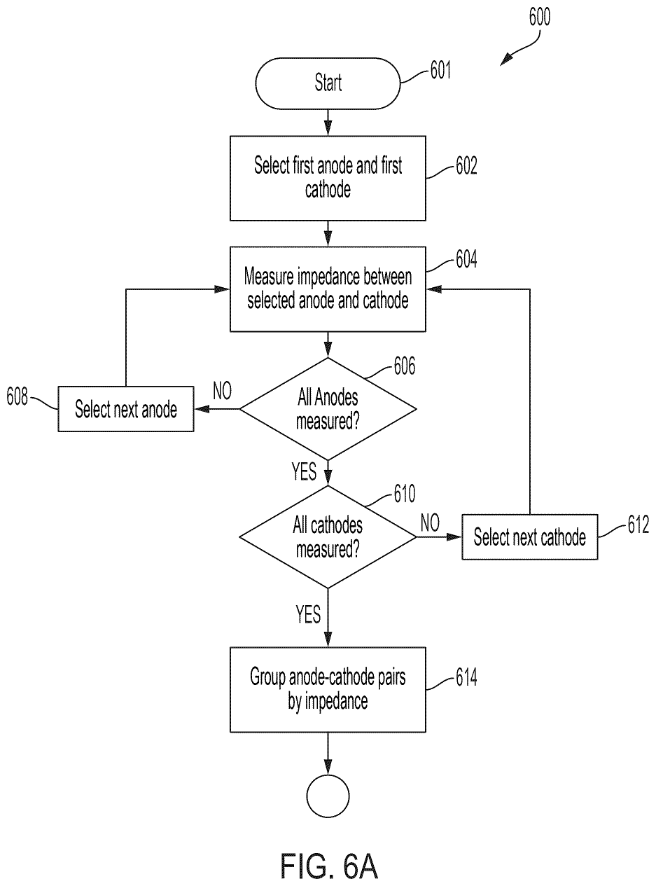

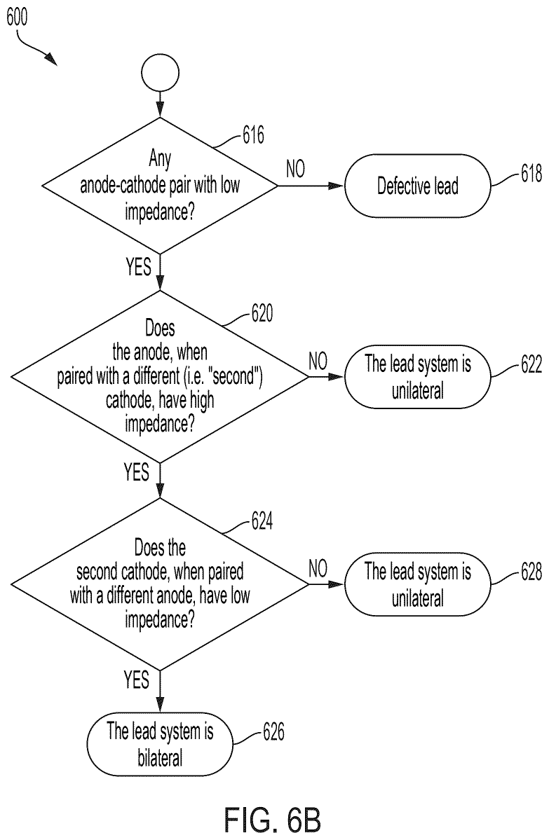

Referring to FIGS. 6A and 6B, a flow diagram is provided to illustrate the automatic lead configuration detection method according to some examples. FIGS. 6A and 6B show two parts of a single flowchart. The method of FIGS. 6A and 6B may be performed or implemented by the processor of the IPG 12, for example, in response to executing corresponding instructions stored in the memory of the IPG 12. As an aside, the illustrated method specifically outlines a process that may be used to identify if there are two electrodes (e.g. two nerve cuffs) or only a single electrode (e.g. a single nerve cuff). The method may be used for stimulation leads which are configured to provide bipolar stimulation, wherein an anode and cathode are both provided upon a distal end of a stimulation lead at the electrode (e.g. nerve cuff) interface. However, those skilled in the art will appreciate that variations of the given method could also be implemented to encompass stimulation systems that include one or more stimulation leads configured to provide monopolar stimulation. Monopolar stimulation may be provided, for example, by setting or driving one or more electrode contacts of an electrode on a stimulation lead as one of a cathode or an anode, and setting or driving another conductive element, such as the housing 14 of the IPG 12, as the other one of the cathode or the anode.

With specific reference to FIGS. 6A and 6B, the method generally comprises iteratively grouping pairs of anodes and cathodes and measuring the impedances therebetween. The magnitudes and/or variation in magnitudes of the measured impedances can include information regarding whether the stimulation lead(s) coupled to the IPG 12 are in a unilateral configuration or a bilateral configuration, and also information regarding whether one or more electrode contacts of the stimulation lead(s) are defective or malfunctioning. The IPG 12 (e.g. the processor of the IPG 12) may be configured to detect, based on the magnitudes and/or variation in magnitudes of the measured impedances, whether the stimulation lead has a unilateral configuration or a bilateral configuration, and, in some examples, whether any electrode contacts on the stimulation lead(s) are defective or malfunctioning.

In some examples, each anode is individually and iteratively grouped with each available cathode, and the respective impedances between each anode-cathode pair is recorded and stored for later reference. Once all available anode and cathode pairs have been tested and their respective impedances stored, the recorded data is used to group anode-cathode pairs according to their recorded impedance values. For example, the anode-cathode pairs may be grouped into a plurality of different groups, respectively corresponding to a plurality of impedance ranges, based on their recorded impedance values. The foregoing process can be useful for enumerating each available electrode contact, be it an anode or a cathode, and it can also be useful for identifying if any of the electrode contacts are malfunctioning. For example, this process can be used to identify each electrode (e.g. each nerve cuff) on the stimulation lead(s), associate each electrode contact with a corresponding one of the identified electrode(s), and, in some examples, determine whether each electrode contact is defective or malfunctioning.

In more detail, the process 600 may begin (e.g., be initiated via the processor) at an initiation stage 601. The process 600 may include a first stage 602 of selecting a first anode and a first cathode (i.e. a first anode-cathode pair) from among the electrode contacts of at least one stimulation lead coupled to the IPG 12, and a second stage 604 of measuring an impedance between the selected anode and the selected cathode. This may entail the processor selecting a pair of feedthrough pins in the header 16, corresponding to the selected anode-cathode pair, and causing the stimulation circuitry to provide stimulation to the selected pair of feedthrough pins such that the stimulation is provided between the selected anode-cathode pair. An impedance corresponding to the selected anode-cathode pair can then be measured between the selected anode and the selected cathode as the stimulation is provided between the selected anode and the selected cathode. For example, when the first anode and the first cathode are selected, a first impedance may be measured between the first anode and the first cathode while the stimulation is provided between the first anode and the first cathode. The magnitude of the first impedance can indicate how far the first anode and the first cathode are spaced apart from each other. A relatively small first impedance can indicate that the first anode and the first cathode are close together and, thus, likely on the same electrode (e.g. the same nerve cuff), while a relatively large first impedance can indicate that the first anode and the first cathode are far apart and, thus, likely on separate electrodes (e.g. separate nerve cuffs) positioned on opposite sides of the body.

The process 600 may include a third stage 606 of determining whether impedances have been measured between each pair of the selected cathode and each anode on the stimulation lead(s). If not, then the process may proceed to a fourth stage 608 of selecting another anode, and then the second stage 604 may be repeated. The second stage 604, third stage 606, and fourth stage 608 may form a first loop process that causes impedances to be iteratively measured between the selected cathode and each of the anodes on the stimulation lead(s). For example, after the first impedance has been measured between the first anode and the first cathode, it may be determined at the third stage 606 that there are other anodes for which an impedance with the first cathode have not yet been measured, a second anode may be selected at the fourth stage 608, and a second impedance may be measured between the second anode and the first cathode. This may be repeated until an impedance between the first cathode and every anode on the stimulation lead(s) has been measured.

If, at the third stage 606, it is determined that impedances between the selected cathode and all anodes have been measured, then the process 600 may proceed to a fifth stage 610. The fifth stage 610 may include determining whether impedances have been measured for all of the cathodes on the stimulation lead(s). If not, then the process 600 may proceed to a sixth stage 612 of selecting another cathode, and then the second stage 604 and the first loop process for the newly selected cathode can be repeated. For example, after impedances between the first cathode and all anodes have been measured, it may be determined at the fifth stage 610 that the stimulation lead(s) includes other cathodes for which impedances have not yet been measured, a second cathode may be selected at the sixth stage 612, an impedance between the second cathode and the selected anode may be measured, and the first loop process may be performed for the second cathode to measure impedances between the second cathode and all of the other anodes.

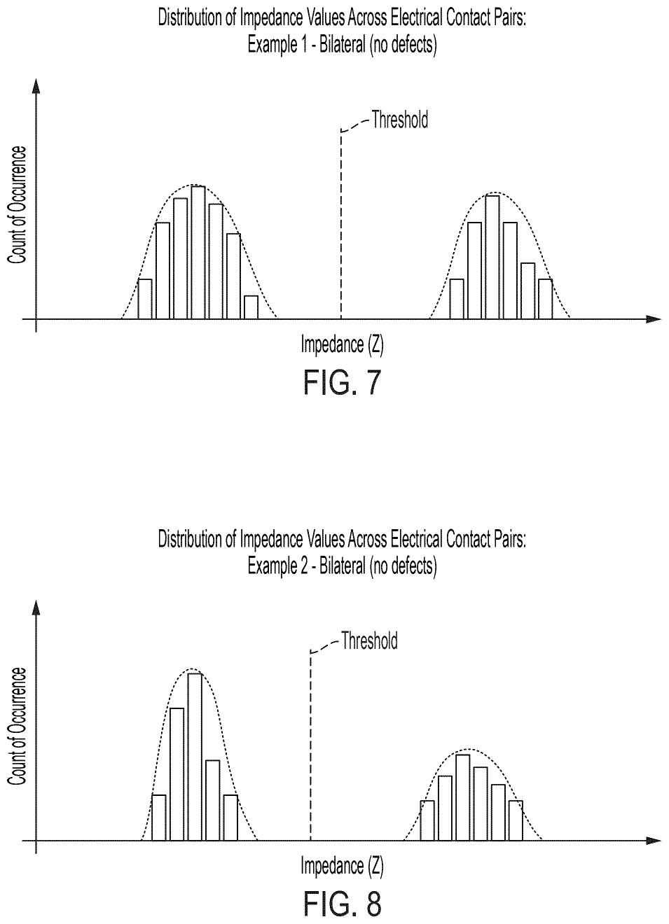

If, at the fifth stage 610, it is determined that impedances have been measured for all of the cathodes of the stimulation lead(s), the process may proceed to a seventh stage 614. During the seventh stage 614, the anode-cathode pairs may be organized into a plurality of groups based on their corresponding measured impedances. For example, the plurality of groups may respectively correspond to a plurality of impedance ranges, and each anode-cathode pair may be assigned to or associated with the group of an impedance range that the anode-cathode's corresponding measured impedance falls within. For example, each group may span a range of 100 Ohms or some other impedance range. In such an example, a first group may correspond to afirst range of 0-100 Ohms, a second group may correspond to a second range of 101-200 Ohms, a third group may correspond to a third range of 201-300 Ohms, etc. An anode-cathode pair having a measured impedance of 250 Ohms would then be assigned to the third group. If multiple anode-cathode pairs each have a measured impedance within the third range, then the third group may have multiple counts of occurrence. When the measured impedances are grouped in this manner, they can be represented via a histogram, similar to the histograms shown in FIGS. 7-11 and discussed in more detail below, and the grouped data can be used to determine if the stimulation lead(s) are in a unilateral configuration or a bilateral configuration and, in some examples, whether any of the anodes or cathodes are defective or malfunctioning. Histograms are described herein as one example of how the anode-cathode pairs can be grouped, and the present disclosure encompasses other ways of grouping the anode-cathode pairs and representing their distribution in view of their measured impedances.

Turning to FIG. 6B, the automatic detection method 600 continues from the seventh stage 614 to an eighth stage 616. During the eighth stage 616, it may be determined if there exists any anode-cathode pair with a measured impedance value that falls below a first threshold (e.g. within a first range having the first threshold as an upper boundary). The first threshold may be any value less than 4000 Ohms. The first range may be, or may be any range subsumed within, 1 to 4000 Ohms, such as about 2000 Ohms to about 4000 Ohms. A typical impedance value between an anode-cathode pair disposed on the same stimulation lead (e.g. disposed on a same electrode, such as a same nerve cuff) is likely to fall within the range of less than 4000 Ohms or between 1.0 to 4000 Ohms. Accordingly, it may be determined that an anode-cathode pair having an impedance less than the first threshold are likely to be on the same electrode. If there does not exist any measured impedance value less than the first threshold, the automated detection program would indicate, at a ninth stage 618, an open circuit, which would indicate a defective stimulation lead, caused by a poorly connected or disconnected conductor in the circuit.

If, at the eighth stage 616, it is determined that there are one or more anode-cathode pairs having an impedance below the first threshold, the process 600 may continue to a tenth stage 620. In the tenth stage 620, for each anode-cathode pair having a relatively low impedance value (i.e. below the first threshold), the program compares the impedance of the anode of the anode-cathode pair (the “subject anode”) with various other cathodes. If the subject anode, when iteratively paired with all other available cathodes, continues to have measured impedance values that all lie below the first threshold value (e.g. below 4000 Ohms), then the program automatically determines at an eleventh stage 622 that the lead system is unilateral. For example, the processor may determine whether the impedance values associated with all other anode-cathode pairs, which include the subject anode, are below the first threshold value. If this is true, it may be determined at the eleventh stage 622 that the lead system has a unilateral configuration.

However, if, at the tenth stage 620, it is determined that there exists an impedance value between the subject anode and at least one other cathode (a “subject cathode”) that is above a second threshold value (e.g. a second impedance range having the second threshold value as a lower boundary), then the program may automatically determine that either (a) the two chosen electrode contacts are in separate electrodes/leads, e.g., that the lead system could be in a bilateral configuration, or that (b) the subject cathode or associated conductor may be electrically malfunctioning or defective, and the process 620 may proceed to a twelfth stage 624. For example, the processor may determine whether the impedance values associated with all other anode-cathode pairs, which include the subject anode, are above the second threshold value. If this is true, the process may proceed to the twelfth stage 624. The second threshold value may be equal to the first threshold value (e.g. 4000 Ohms) or may be different from (e.g. greater than) the first threshold value. For example, the second threshold value may be any impedance value equal to or greater than 4000 Ohms. A relatively high impedance for an anode-cathode pair can indicate that the anode and cathode are spaced relatively far apart from each other with a substantial amount of tissue therebetween (as would be the case in a bilateral configuration), or that the cathode is malfunctioning or defective.

The twelfth stage 624 may include iteratively grouping the subject cathode with different anodes. If it is determined that the subject cathode, when paired with a different anode, has a relatively low impedance (e.g. below the first threshold), then it may be determined at a thirteenth stage 626 that the second cathode is not malfunctioning and the lead system is automatically determined to be bilateral.

On the other hand, if it is determined at the twelfth stage 624 that there does not exist an anode-cathode pair that includes the subject cathode and that has a measured impedance that lies below the first threshold, then the automatic detection method may determine at a fourteenth stage 628 that the lead system is unilateral and that the subject cathode is malfunctioning. This may be based on the assumption that an electrode having a cathode will also have at least one anode. Thus, if there is an anode-cathode pair that includes the subject cathode and that has an impedance below the first threshold, it may be determined that the subject cathode and the anode of the anode-cathode pair are nearby and, thus, on the same electrode (e.g. the same nerve cuff). Accordingly, it can be determined at that stage that the stimulation lead system includes at least two electrodes (e.g. at least two nerve cuffs), and that the stimulation lead system is in a bilateral configuration. However, if there is no such anode-cathode pair, then it may be determined that the subject cathode is merely defective or malfunctioning.

With returned reference to FIG. 4 in light of the method described in FIGS. 6A and 6B, a first measured impedance “ZA” is measured between a first cathode and a first anode, which are both disposed on a first nerve cuff 202, and a second measured impedance “ZB” is measured between the first cathode and a second anode, which are disposed on a separate nerve cuffs on opposing sides of the neck. In practice, the first impedance ZA would be relatively lower than the second impedance ZB, and, therefore, the method outlined in FIGS. 6A and 6B may be utilized to automatically determine that the lead system is configured for bilateral stimulation.

It should be noted that the foregoing method is described simplistically as being applied to only a handful of available anodes and cathodes for the purpose of illustrating the general method by which the automatic detection algorithm can determine if a lead system is unilateral or bilateral, it being understood that the steps of iteratively grouping various anodes and cathodes and comparing the resulting impedance values could, in practice, be repeated for each and every available anode-cathode sub combination.

With reference to FIGS. 7-11, the various histograms provide exemplary distributions of impedance values that are iteratively measured across pairs of electrode contacts within connected leads, wherein each and every electrode contact is included within at least one measurement. These histograms illustrate how many pairs of electrode contacts (i.e. the “Count of Occurrence” on the vertical axis) are grouped within each of a plurality of groups respectively associated with a plurality of impedance ranges based on the measured impedance values of the pairs of electrode contacts. FIGS. 7-11 illustrate how the automated lead configuration detection algorithm collectively analyzes the measured impedance values, groups the impedance values according to their relative impedances, and dynamically applies one or more threshold values to distinguish between groups of measured impedance values for the purpose of determining whether the connected lead(s) are unilateral or bilateral, and, in some examples, for detecting if any of the connected leads contain electrically defective elements. For example, a histogram containing a unimodal distribution of measured impedance values may be indicative of a unilateral lead configuration, whereas a bimodal distribution of impedance values may be indicative of a bilateral lead configuration. Further, very high impedance values (i.e. lying outside of unimodal or bimodal distribution) would be indicative of one or more defective electrodes (e.g. an open circuit), and very low impedance values would be indicative of an electrical short within the stimulation lead. For example, if it is determined that the impedance value of each of one or more anode-cathode pairs, which include a common electrode contact, is above a third threshold value greater than the second threshold value, it may be determined that the common electrode contact is defective due to, for example, an open circuit. The third threshold value may be any impedance value greater than or equal to 8000 Ohms, such as 10000, 15000, 20000, or 30000 Ohms. If it is determined that the impedance values of each of one or more anode-cathode pairs, which include a common electrode contact, is below a fourth threshold value less than the first threshold value, it may be determined that the common electrode contact is defective due to an electrical short within the stimulation lead(s). The fourth threshold value may be any impedance value less than or equal to 2000, such as 1500, 1000, or 500.

Using a dynamic threshold detection approach, the automated lead configuration detection algorithm can further determine whether a given lead includes a single nerve cuff or multiple nerve cuffs, even when two or more nerve cuffs are implanted on the same side of the body, be they implanted upon the same nerve at distinct locations (e.g. proximal hypoglossal nerve and distal hypoglossal nerve), or upon two different nerves on the same side of the body (e.g., one nerve cuff on the hypoglossal nerve to treat OSA, and another nerve cuff on the vagus nerve to treat epilepsy).

Due to relative differences between (a) bio-impedance values measured between electrode contacts disposed within the same nerve cuff, (b) bio-impedance values measured between electrode contacts disposed on separate nerve cuffs on the same side of the body (which are separated by some tissue and therefore have relatively higher impedances), and (c) bio-impedance values measured between electrode contacts disposed on separate nerve cuffs on different sides of the body (which are separated by much more tissue, and therefore have substantially higher impedances), the algorithm can quantitatively determine the number of electrode interfaces (e.g. nerve cuffs) on a given lead and further determine if the leads are configured to provide stimulation unilaterally or bilaterally.

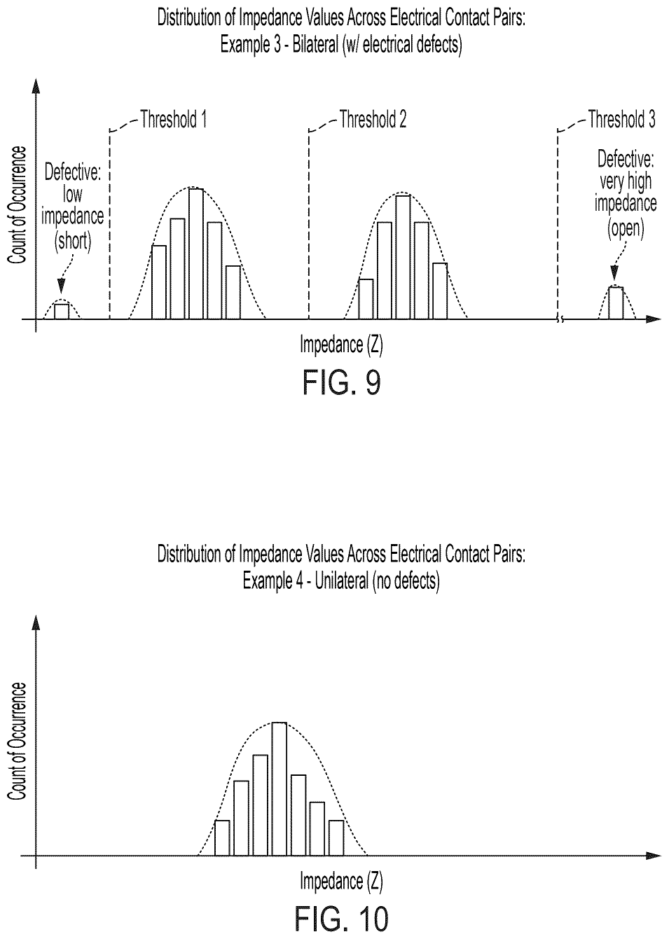

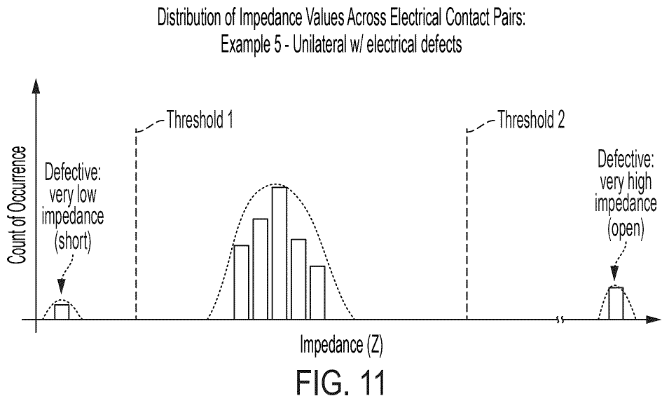

Generally referring to FIGS. 7-9, each of the illustrated histograms contain a primarily bimodal distribution of measured impedance values. As such, in each instance, the automated lead configuration detection algorithm would indicate that the connected leads are configured to provide stimulation bilaterally. Further, with specific reference to FIG. 9, the algorithm may automatically detect outlying impedance values that are indicative of electrical defects within the stimulation lead(s). In FIG. 9, anode-cathode pairs having impedance values between Threshold 1 and Threshold 2 may indicate properly functioning pairs of electrode contacts on a first electrode (e.g. a first nerve cuff), anode-cathode pairs having impedance values between Threshold 2 and Threshold 3 may indicate properly functioning pairs of electrode contacts on a second electrode (e.g. a second nerve cuff) positioned on an opposite sagittal side of the body from the first electrode, anode-cathode pairs having an impedance above Threshold 3 may indicate pairs of electrode contacts that include a defective electrode contact (e.g. due to an open circuit), and anode-cathode pairs having an impedance below Threshold 1 may indicate pairs of electrode contacts that include a defective electrode contact (e.g. due to an electrical short). Turning to FIGS. 10-11, each of the illustrated histograms contain a unimodal distribution of measured impedance values. Thus, in each instance, the automated lead configuration detection algorithm would indicate that that the connected leads are configured to apply stimulation unilaterally. Further, with reference to FIG. 11, the algorithm has automatically detected outlying impedance values that are indicative of electrical defects within the stimulation lead(s). In FIG. 11, anode-cathode pairs having impedance values between Threshold 1 and Threshold 2 may indicate properly functioning pairs of electrode contacts on an electrode (e.g., a nerve cuff), anode-cathode pairs having impedance values above Threshold 2 may indicate pairs of electrode contacts that include a defective electrode contact (e.g., due to an open circuit), and anode-cathode pairs having impedance values below Threshold 1 may indicate pairs of electrode contacts that include a defective electrode contact (e.g. due to an electrical short).

In some examples, the processor of the IPG 12 may be configured to determine the stimulation lead configuration and/or whether any of the electrode contacts are defective based on the variation of the impedance values of the anode-cathode pairs, for example, as measured in the first to sixth stages 602 to 612 of the process 600. For example, the processor may be configured to determine whether the impedance values are arranged in a unimodal distribution or in a bimodal distribution. This may include, for example, grouping the anode-cathode pairs according to the seventh stage 614, for example, to generate a histogram or other distribution representation, and determining, based on the grouping (e.g., based on the histogram or other distribution representation) that the impedance values are in a unimodal or bimodal arrangement. The processor may be configured to determine that the lead system is in a unilateral configuration in response to determining that the impedance values are in the unimodal distribution, and to determine that the lead system is in a bilateral configuration in response to determining that the impedance values are in the bimodal distribution. The processor may also be configured to determine, for example, based on the grouping of the impedance values, that one or more electrode contacts are defective.

After automatically detecting the configuration of attached stimulation lead(s) 100, 200, 204, 205, 300, 304, and/or 305, the IPG 12 may be configured to wirelessly transmit (e.g. via a transmitter or transceiver) information pertaining to the detected lead configuration to the clinicians programming device 20 and/or automatically implement an IPG workflow configuration that works congruously with the detected lead configuration. In some embodiments, information pertaining to the detected lead configuration may be displayed on a graphical user interface of the clinicians programming device 20 (FIG. 1) and/or the patient programmer 22 (FIG. 1), for example, in response to receiving such information. Thus, the automated lead configuration detection process drastically reduces the potential for errors which might otherwise occur when programmatically configuring a newly implanted IPG 12 to function in accordance with a given lead configuration.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes examples having the features recited in each dependent claim in combination with the features recited in every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A method for automatically detecting a lead configuration of one or more implanted stimulation leads, the method comprising:
applying stimulation through a first pair of electrode contacts of the one or more stimulation leads and measuring a first impedance value between the first pair;
applying stimulation through a second pair of electrode contacts of the one or more stimulation leads and measuring a second impedance value between the second pair;
comparing the first impedance value and the second impedance value to one or more threshold values; and
determining, based on the comparison of the first impedance value and the second impedance value to the one or more threshold values, if the lead configuration is a unilateral configuration or a bilateral configuration.

2. The method of claim 1, wherein one electrode contact is common between the first and second pairs of electrode contacts.

3. The method of claim 2 further comprising applying stimulation through a third pair of electrode contacts of the one or more stimulation leads and measuring a third impedance value between the third pair.

4. The method of claim 3, wherein one electrode contact is common between the second and third pairs of electrode contacts.

5. The method of claim 1, wherein the one or more stimulation leads are electrically coupled to an implantable medical device.

6. The method of claim 5, wherein the implantable medical device is an implantable pulse generator.

7. The method of claim 1 further comprising determining whether any of the electrode contacts of the first and second pairs are defective or malfunctioning based on the comparison of the first impedance value and the second impedance value to the one or more threshold values.

8. The method of claim 1, further comprising displaying an indication pertaining to the determined lead configuration on a graphical user interface.

9. The method of claim 8, further comprising wirelessly transmitting data pertaining to the determined lead configuration to an external controller.

10. The method of claim 1, wherein the one or more threshold values includes a first threshold value within a range of about 2000 Ohms to about 4000 Ohms.

11. The method of claim 10, further comprising generating an alert indicating a detected lead fault if one or more of the first impedance value and the second impedance value are greater than a second threshold value greater than the first threshold value.

12. The method of claim 1, wherein the lead configuration is determined to be the unilateral configuration in response to determining that both the first and second impedance values are less than a first threshold value.

13. The method of claim 1, wherein the lead configuration is determined to be the bilateral configuration in response to determining that the first impedance value is less than a first threshold value, and the second impedance value is greater than a second threshold value.

14. The method of claim 13, wherein the first and second pairs of electrode contacts share a first electrode contact,
wherein the method further comprises:
applying stimulation through a third pair of electrode contacts of the one or more stimulation leads and measuring a third impedance value between the third pair, the second and third pairs of electrode contacts sharing a second electrode contact different from the first electrode contact; and
comparing the third impedance value to the one or more threshold values, and
wherein the lead configuration is determined to be the bilateral configuration further in response to determining that the third impedance value is less than the first threshold value.

15. An implantable stimulation system comprising:
an implantable pulse generator;
at least one implantable stimulation lead proximally connected to the implantable pulse generator and distally connected to at least one neural interface; and
a processor configured to automatically determine a lead configuration of the at least one implantable stimulation lead to be a unilateral configuration or a bilateral configuration.

16. The implantable stimulation system of claim 15, wherein the processor is configured to iteratively measure impedance values between pairs of electrode contacts disposed on the at least one neural interface.

17. The implantable stimulation system of claim 16, wherein the processor includes a memory configured to store the measured impedance values between the pairs.

18. The implantable stimulation system of claim 16, wherein the processor is configured to determine the lead configuration based on the measured impedance values between the pairs.

19. The implantable stimulation system of claim 18, wherein the processor is configured:
to determine whether a distribution of the measured impedance values is unimodal or bimodal; and
to determine the lead configuration based on whether the distribution is unimodal or bimodal.

20. The implantable stimulation system of claim 18, wherein the processor is configured:
to determine whether each of the measured impedance values is below or above a first threshold value; and
to determine that the lead configuration is the unilateral configuration in response to determining that all of the measured impedance values are below the first threshold value.

21. The implantable stimulation system of claim 20, wherein the first threshold value is equal to or less than 4000 Ohms.

22. The implantable stimulation system of claim 20, wherein the processor is configured:
in response to determining that the impedance of a first pair of the pairs of electrode contacts is above the first threshold, to determine whether a second pair of the pairs of electrode contacts exists that both includes a first electrode contact that is also included in the first pair and also has an impedance value below the first threshold value; and
to determine that the lead configuration is the bilateral configuration in response to determining that such a second pair exists.

23. The implantable stimulation system of claim 16, wherein the processor is configured:
to determine whether each of the measured impedance values is below or above a second threshold value, the second threshold value being greater than 20,000 Ohms; and
in response to determining that one or more of the pairs of the electrode contacts, each including a common first electrode contact, has an impedance value above the second threshold value, to determine that the common first electrode contact is defective.

24. The implantable stimulation system of claim 15, wherein the processor is configured to wirelessly transmit data pertaining to the determined lead configuration to a clinician programmer.

25. The implantable stimulation system of claim 15, wherein the processor is configured to display data pertaining to the determined lead configuration on a graphical user interface.

26. The implantable stimulation system of claim 15, wherein the at least one implantable stimulation lead comprises a bifurcated lead body.

27. The implantable stimulation system of claim 15, further comprising a patient programmer configured to communicatively couple to the implantable pulse generator.

28. The implantable stimulation system of claim 15, wherein the at least one implantable stimulation lead is configured to apply stimulation upon at least one of the hypoglossal nerve, the vagus nerve, or another cranial nerve.

29. An implantable pulse generator comprising:
stimulation circuitry;
a lead receptacle configured to receive an end of a stimulation lead;
feedthrough pins coupled between the stimulation circuitry and the lead receptacle; and
a processor operatively coupled to the stimulation circuitry and configured:
for each of a plurality of different pairs of the feedthrough pins, to apply stimulation to the pair of feedthrough pins via the stimulation circuitry and to measure an impedance between the pair of feedthrough pins, and
to determine a lead configuration of the stimulation lead based on the plurality of measured impedances respectively corresponding to the plurality of different pairs of feedthrough pins.

30. The implantable pulse generator of claim 29, wherein the processor is configured to determine the lead configuration to be a unilateral configuration or a bilateral configuration based on the plurality of measured impedances.

31. The implantable pulse generator of claim 29, wherein the processor is configured to compare each of the plurality of measured impedances to one or more threshold impedance values, and to determine the lead configuration based on the comparison of the plurality of measured impedances to the one or more threshold impedance values.

32. The implantable pulse generator of claim 29, wherein the processor is configured to determine the lead configuration based on a distribution of the plurality of measured impedances.
